(19) United States
(12) Patent Application Publication
(10) Pub. No.: US 2022/0176116 A1
(43) Pub. Date: Jun. 9, 2022

(54) ELECTRO-STIMULATION APPARATUS EFFECTIVE IN AURICULAR STIMULATION

(71) Applicant: Stoparkinson Healthcare Systems, LLC, Fort Lauderdale, FL (US)

(72) Inventors: O. Burak Özdoganlar, Sewickley, PA (US); Yusuf Özgür Çakmak, Istanbul (TR); Burak Özsoy, Istanbul (TR); Yalin Geçer, Istanbul (TR); Ali Cem Soysal, Istanbul (TR)

(73) Assignee: Stoparkinson Healthcare Systems, LLC, Fort Lauderdale, FL (US)

(21) Appl. No.: 17/545,712

(22) Filed: Dec. 8, 2021

Related U.S. Application Data
(60) Provisional application No. 63/122,517, filed on Dec. 8, 2020.

(51) Int. Cl. A61N 1/36; A61M 37/00; A61N 1/372; A61N 1/05

(52) U.S. Cl. CPC A61N 1/36003; A61M 37/0015; A61N 1/36036; A61N 1/36153; A61M 2037/0023; A61N 1/37205; A61N 1/0502; A61M 2037/0061; A61N 1/36171

(57) ABSTRACT

An electro-stimulation apparatus provides stimulation of the supplementary motor area, premotor area, cerebellum and/or subthalamic nucleus of a human. The electro-stimulation apparatus includes at least one micro needle electrode having a stimulation end and a base. Said at least one micro needle electrode is provided with a stimulation end configured to stimulate intrinsic auricular muscles of the human and said stimulation end of said micro needle electrode is adapted to generate an electrical stimulation signal during a stimulating state.

### PRIORITY CLAIM

This application claims priority to U.S. provisional application Ser. No. 63/122,517, filed Dec. 8, 2020, which is entirely incorporated by reference.

### TECHNICAL FIELD

The present disclosure relates to an electro-stimulation apparatus where the supplementary motor area, premotor area, cerebellum and/or subthalamic nucleus are stimulated.

### BACKGROUND

Abnormal resting overactivity as tremors can be caused by various conditions or medicines that affect the nervous system, including Parkinson's disease (PD), liver failure, alcoholism, mercury or arsenic poisoning, lithium, and certain antidepressants. Rigidity, bradykinesia and postural instability are some of the other symptoms of Parkinson's disease besides tremors. Parkinson's disease is a chronic and progressive movement disorder, meaning that symptoms continue and worsen over time. According to European Parkinson's Disease Association, it is estimated that 6.3 million people in the world are living with Parkinson's disease. The cause is unknown, and although there is presently no cure, there are treatment options such as medication and surgery to manage its symptoms.

### SUMMARY

Stimulation of different parts of a brain with different techniques can be successfully used for the treatment of Parkinson's disease.

The main objectives of deep brain stimulation (DBS) devices are electrical stimulation of the subthalamic nucleus and, as a consequence, activation of the supplementary motor areas and premotor areas and normalization of the abnormal resting overactivity in the motor system.

Subthalamic Nucleus-Deep Brain Stimulation (STN-DBS) is an invasive but effective approach to alleviating Parkinson's disease (PD) Symptoms. Standard STN-DBS for PD is usually delivered 100 Hz to 250 Hz (130 Hz-185 Hz) with a voltage level of 1-4V and pulse width of 60 microseconds. On the other hand, to achieve specific effects, different frequencies are generally used; for instance, although 60 Hz is considered effective for improving swallowing, freezing and axial gait functions, 130 Hz is not effective. Moreover, for verbal fluency, 60 Hz works better than 130 Hz. For the tremor-resistant patients, the frequency is generally selected as 180 Hz. Based on the available data, a particular stimulation frequency may be needed for alleviating different symptoms of PD.

Current applications to stimulate the subthalamic nucleus include intracranial electrode placement, which is referred to as deep brain stimulation (DBS). The process of deep brain stimulation of the subthalamic nucleus requires a neurosurgery, which is an extremely invasive intervention for Parkinson's patients. In this neurosurgery operation, electrodes are placed into the subthalamic nucleus region that connects with all the muscles of the human body. The neurons in this region receive feedback (like a stretch) from the muscles. In other words, to stimulate the STN externally, a nerve that is related to muscle innervation should be stimulated.

Further, surgical device applications are likely to have side effects. Moreover, the stimulator's battery is placed under the thorax skin while the electrodes are inserted into the brain tissue, and the wires go under the skin. The frequency and the intensity of these simulators can be altered wirelessly with an external unit. The United States patent application U.S. Pat. No. 5,707,396 discloses a method of arresting the degeneration of the substantia nigra by high-frequency stimulation of the subthalamic nucleus. This method requires neurosurgical implantation of the electrodes into substantia nigra and surgical implantation of a battery.

Among others, a prior art publication in the technical field of the present disclosure is U.S. Pat. No. 5,514,175, which discloses a low voltage, multi-point auricular stimulator device that treats dysfunctions in neural pathways by acting upon multiple auricular points. Another reference is the European patent disclosure EP 2474339, disclosing a resuscitation device for resuscitation by stimulating an auricle of the ear. Further prior art references in the present technical field include WO2014207512A1, US 2008/0249594, US 2013/0079862, or WO 2010/048261.

The electro-stimulation apparatus disclosed herein addresses the situation where an extra-cranially placed device not only controls the symptoms of Parkinson's but also reduces the level of pain. The design also improves the perception of the device and the patient's compliance and response to the stimulation. To this end, the present electro-stimulation apparatus proposes an electrode placed on the intrinsic muscles for effectuating stimulation of the intrinsic auricular muscles in multiple locations with appropriate frequency combinations to obtain symptom-specific results.

Different techniques provide methods for accurately locating the intrinsic auricular muscles connected to the predefined regions of the brain with neuropathy channels. EMG (Electromyography) sensors measure electrical currents/impulses generated in muscles during the contraction thereof and at rest. The present electro-stimulation apparatus is devised under the recognition that the collected data by the EMG sensor is used to drive the stimulator and adjust various settings during the effecting of the muscle stimulation.

### Interesting Features of the Electro-Stimulation Apparatus

An interesting feature of the electro-stimulation apparatus is using micro-scale needle electrodes for improved clinical deployment.

Another interesting feature of the electro-stimulation apparatus is the minimization of pain during the insertion of micro needle electrodes.

Another interesting feature of the electro-stimulation apparatus is the elimination of the patient's perception of the painful application.

Another interesting feature of the electro-stimulation apparatus is easy administration and removal of micro needle electrodes.

Another interesting feature of the electro-stimulation apparatus is minimization of tissue damage during insertion, removal, and reapplication.

Another interesting feature of the electro-stimulation apparatus is maintaining the electrodes securely in place for an extended duration.

Another interesting feature of the electro-stimulation apparatus is the provision of sufficient conductivity and a straightforward regulatory pathway.

Another interesting feature of the electro-stimulation apparatus is efficient manufacturability with a high level of reproducibility and low cost.

Another interesting feature of the electro-stimulation apparatus is the provision of sterilizable micro needles.

Another interesting feature of the electro-stimulation apparatus is the provision of a low impedance connection for the electrical leads.

### BRIEF DESCRIPTION OF THE TECHNICAL DRAWINGS

Accompanying drawings are given solely for the purpose of exemplifying an electro-stimulation apparatus, whose advantages over prior art were outlined above and will be explained briefly hereinafter.

The drawings are not meant to delimit the scope of protection as identified in the Claims, nor should they be referred to alone in an effort to interpret the scope identified in said Claims without recourse to the technical disclosure herein.

FIG. 1 demonstrates a schematic view of an example of separated microneedles and a base of an electro-stimulation apparatus.

FIG. 2 demonstrates a schematic view of an electro-stimulation apparatus in which micro needle electrodes and the base are fabricated together according to an example.

FIG. 3 demonstrates a schematic view of a magnet incorporated into the metal base according to an example.

FIG. 4 demonstrates a schematic view of an electro-stimulation apparatus in which micro needle electrodes include undercutting features.

FIG. 5 demonstrates a schematic view of an electro-stimulation apparatus in which undercut coating applied according to an example.

FIG. 6 demonstrates a schematic view of an electro-stimulation apparatus with an adhesive layer applied on the base according to an example.

### DETAILED DESCRIPTION

The following numerals are referred to herein:

10) Electro-stimulation apparatus

11) Stimulation end

12) Magnetic element

13) Base

14) Conductive backing

15) Undercut

16) Adhesive layer

17) Dissolvable undercut element

18) Micro needle electrode

19) Control Unit

20) Communication Unit

Examples of the present electro-stimulation apparatus (10) includes at least one micro needle electrode (18). Each micro needle (18) includes a stimulation end (11) for controlling the stimulation process based on the collected data. Said micro needle electrodes (18) are inserted through the auricular skin such that the stimulation end (11) of the electrode reaches specific intrinsic auricular muscles of a human ear.

At least one micro needle electrode (18) of the electro-stimulation apparatus (10) is adapted to be directly attached to intrinsic auricular muscles such as helicis major muscles, helicis minor muscles, tragicus muscles, anti-tragicus muscles. According to an example, the tragicus, anti-tragicus, and helices minor muscles of the ear are stimulated with a pulse signal at a specific frequency (1 Hz-1 kHz, e.g., 130 Hz), pulse width (1 ps-1,000 e.g., 100 ps). The voltage (1-10 V) is selected by the physician, below the pain threshold of a patient. One of the probes is used as the anode and the other as the cathode; the same signal patterns are applied to each probe, except with a phase shift equal to one-half of the pulse period.

According to another example feature, said micro needle electrodes (18) have 2 mm height, 50-750 μm diameter and have a surgical stainless-steel (ASTM 430F) or Ti6Al4V core. Optionally, they can be gold plated, with or without a copper layer between the core material and gold.

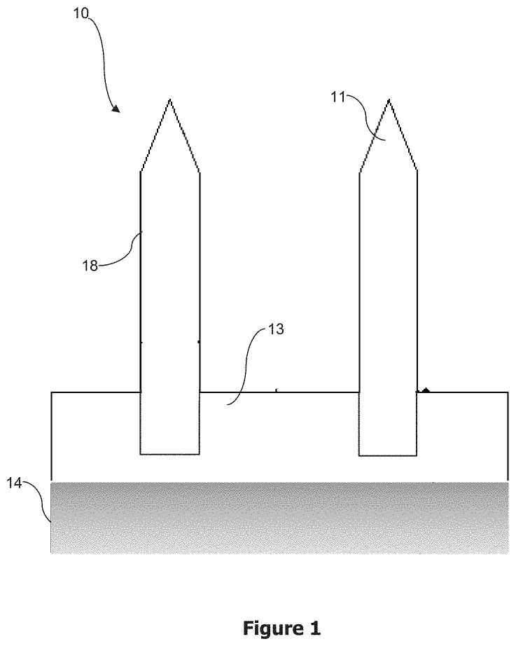

In reference to FIG. 1, the micro needle electrodes (18) are connected to a base (13), which remains on top of the skin after the insertion process. Said base (13) can be made of any metal. According to the example, said base (13) is assembled with the micro needle electrodes (18) using, for instance, a conductive adhesive or a mechanical connection. In an example feature, the connection between the base (13) and the micro needle electrodes (18) is established through a screw action. The helical ridge on the micro needle electrodes (18) and the thread on the base (13) enable electrical communication and also a type of connection which is detachable as a mechanical connection. The microneedles and the base (13) create a monolithic structure. When the micro needle and the base are made from a non-ferromagnetic material (such as titanium alloys), another base (14) is attached to the bottom. This base will provide ferromagnetic properties while still being an electrical conductor, and facilitates the attachment of leads onto the probes using magnets. A conductive backing (14) is included to enable attaching the electrical leads onto the back of the microneedle electrode assembly. In this example, said conductive backing (14) can be magnetic, either including a natural magnet, or a ferromagnetic material onto which a magnet can be attached. The removable micro needle electrode (18) structure provides an effortless maintenance process for the electro-stimulation apparatus (10). Further, said base (13) is reusable in the case of removal of the micro needle electrodes (18) for any reason.

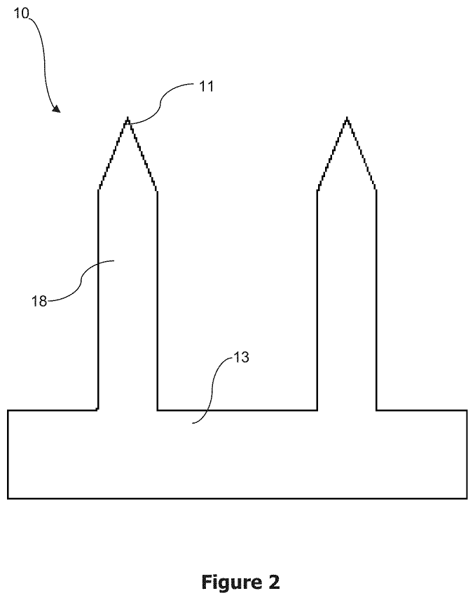

FIG. 2 shows an example apparatus (10) in which the base (13) is monolithically integrated with the micro needle electrodes (18). Integrated structure of micro needle electrodes (18) and the base (13) provides a simpler fabrication process. In this example, the micro needle electrodes (18) are made of Ti6Al4V. Each needle has a height of 2 mm, and the base has a thickness of 2 mm. Micro-endmills can be used on customized, high-precision, miniature machining systems to remove the material from the desired locations to create the micro needle electrodes (18). After fabrication, the micro needle electrodes (18) should be measured to assess precision and reproducibility. The advantage of this approach is that no assembly with a base (13) is needed as the base (13) is already monolithically integrated with the micro needle electrodes (18).

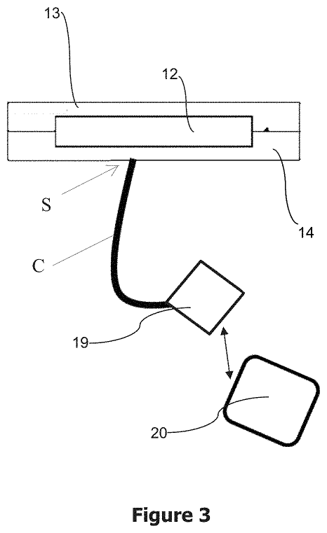

FIG. 3 shows an example apparatus (10) where a magnetic element (12) is incorporated into the metal base (13). Said magnetic element (12) is sandwiched between a conductive backing (14) and a metal base (13). In this example, any cable (C) to transmit pulse signals connects to the metal base (13) with ease. Furthermore, if any accident occurs during the transmission process, the cable connection will break immediately to prevent any tissue damage. In other words, the cable (C) is held in place magnetically so that if it is tugged, it will pull out of the socket (S) without hurting the patient or damaging the tissue, and without pulling the connected control unit (19) off the skin surface on which it is located. Also, the magnetic grip force provides a stable and effortless connection.

In the example of FIG. 3, a control unit (19) that includes controller circuitry, in communication with a communication unit (20) that includes communication circuitry are illustrated. The controller circuitry of the control unit (19) may include one or more processors and memory. The memory stores, for example, instructions that the processor(s) executes to carry out desired functionality for the apparatus (10). Control parameters stored in memory may provide and specify configuration and operating options for the instructions. For instance, the control instructions and control parameters may implement all or a portion of the functionality described herein. The memory 120 may also store data, such as data, that the apparatus (10) has generated and/or will send, or has received by the control unit (19), through the communication unit (20).

The communication unit (20) may include wireless transceiver circuitry, e.g., radio frequency (RF) transmit (Tx) and receive (Rx) circuitry, to perform transmission and reception of signals through one or more antennas. Accordingly, the wireless transceiver circuitry may include modulation/demodulation circuitry, digital to analog converters (DACs), shaping tables, analog to digital converters (ADCs), filters, waveform shapers, filters, pre-amplifiers, power amplifiers and/or other logic for transmitting and receiving through one or more of the antennas. The communication unit (2) may also include wired or physical medium transceiver circuitry. Examples of physical media include optical fiber, coaxial cable such as RG6, telephone lines, network (e.g., Ethernet) cables, buses such as the PCIe bus, and serial and parallel cables. Accordingly, the physical medium transceiver circuitry may include Tx and Rx circuits for communication according to, as examples, Ethernet, asynchronous transfer mode (ATM), data over cable service interface specification (DOCSIS), Ethernet passive optical network (EPON), EPON protocol over coax (EPoC), synchronous optical networking (SONET/SDH), multimedia over cable alliance (MoCA), digital subscriber line (DSL), over associated physical media. As such, the signals transmitted and received by the communication unit (20) may adhere to any of a diverse array of formats, protocols, modulations (e.g., QPSK, 16-QAM, 64-QAM, or 256-QAM), frequency channels, bit rates, and encodings.

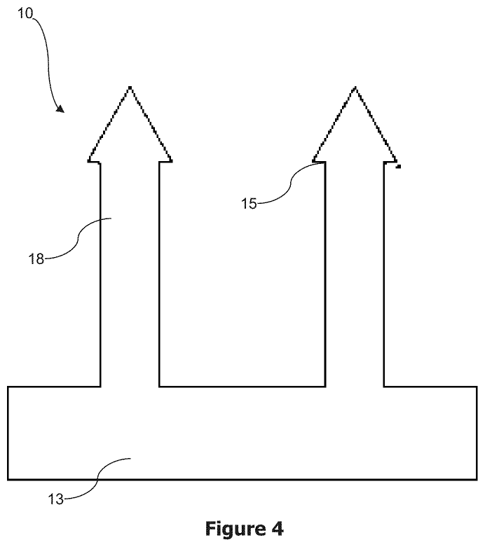

FIG. 4 shows an example apparatus (10) in which micro needle electrodes (18) have undercut features. Said undercuts (15) are formed to retain micro needle electrodes (18) in place. During the long-term usage of the electro-stimulation apparatus (10), the relative motion between the micro needle electrodes (18) and the tissue must be minimized to ensure minimal or no tissue damage. Furthermore, a robust attachment may also be a requirement to provide consistent stimulation currents to the auricular intrinsic muscle zones. Said undercuts (15) provide retaining features on the micro needle electrodes (18) themselves. According to an example, there is a type of undercut to form an arrowhead-shaped stimulation end (11). Similarly, undercuts (15) can be formed away from the stimulation end (11). In an example, a combination of the two aforementioned approaches can be used: small undercut features, smaller than when used singly, can be combined with an adhesive tape to ensure effective fixturing of the micro needle electrodes (18) while minimizing the tissue damage and pain.

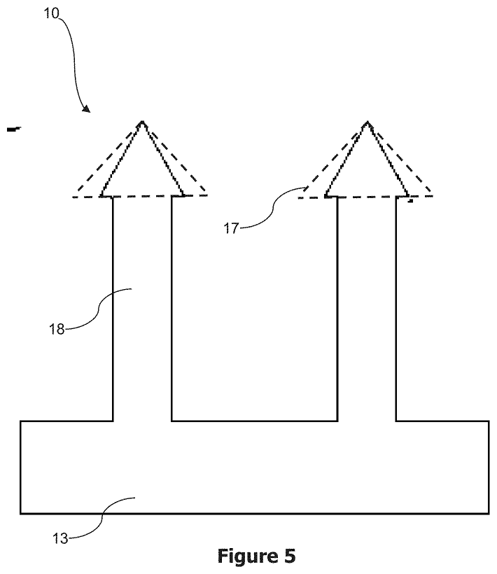

FIG. 5 shows an example apparatus (10) in which a dissolvable undercut element is (17) attached to the micro needle electrodes (18). Said dissolvable undercut element (17) is made of a biocompatible and dissolvable material, such as simple or complex sugars, polyvinyl alcohol, or polylactic-co-glycolic acid (PLGA). The dissolvable undercut element (17) expands said undercut (15) dimensions to be located firmly under any tissue. The water, heat and/or the organic content in the tissue enable said dissolvable undercut element (17) to dissolve over time. In an example, whole undercut (15) is a dissolvable undercut element (17), therefore when the dissolvable undercut element (17) is dissolved completely, there is no undercut (15) feature left to hold micro needle electrode (18) under the tissue. In an example, said dissolvable undercut element (17) is a dissolvable arrow-head shaped tip with undercutting form. In another example, the dissolvable element is formed by coating or molding. Those forms provide retaining capability to securely fix the electro-stimulation apparatus (10) in place for the duration of usage. For this purpose, the stimulation end (11) materials can be selected from dissolvable materials, such as PLGA, where the dissolution time can be selected. This can be extended to weeks by changing the polylactic to glycolic acid ratio. The approach here is that the stimulation ends (11) and their undercuts will be dissolved fully or almost entirely when it is time to retract the micro needle electrodes (18). Therefore, easier, pain-free, and tissue-damage-free extraction can be achieved. In a further example, said dissolvable undercut element (17) encapsulates anti-inflammatory and/or local anesthetics and/or painkiller substances. Also, said dissolvable undercut element (17) comprises materials that can be dissolved under determined electrical stimulation frequency. Said dissolvable undercut element (17) can comprise any drug content. Thus, drug release with the desired frequency is initiated in the target area. For instance, hydrogen sulfide has a gaseous neurotransmitter role in Parkinson's branch brain networks and is also a neuroprotector. In another example, micro needle electrodes (18) have different sizes to affect individual locations depending on the depth and location. Even, in the array of micro needle electrodes (18) on the same base (13), particular micro needle electrode (18) size can vary.

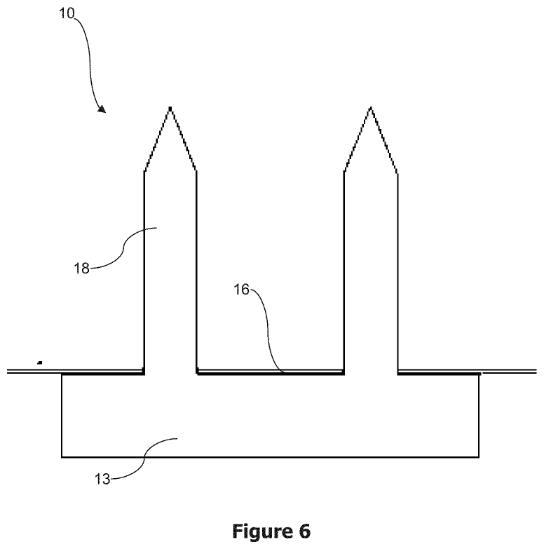

FIG. 6 shows an example apparatus (10) in which an adhesive layer (16) is used on the metal base (13). Said adhesive layer (16) ensures effective fixturing of the micro needle electrodes (18) while minimizing tissue damage and pain. Various options can be combined to immobilize the micro needle electrodes (18). In an example, undercut (15) features can be combined with the adhesive layer (16) on the metal base (13). This combination enables using smaller undercuts (15) to minimize the pain with the cooperation of the adhesive layer (16). In another example, said undercuts (15) are dissolvable elements.

In an example, micro needle electrodes (18) are assembled onto a metallic base (13). The stimulation end (11) of said micro needle electrodes (18) must be appropriate for insertion into a human ear. The sharpening process may be accomplished by acid etching the stimulation end (11). Said micro needle electrodes (18) will then have to be assembled onto the metal base (13) through, e.g., soldering or conductive adhesives.

In another example, bent-out micro needle electrodes (18) from metal sheets can be used. In this method, the profile of the micro needle electrodes (18) is cut out of a metal sheet (e.g., using mechanical micromachining or laser cutting), and then a fixture is used to bend out the micro needle electrodes (18) to create projections.

In another example, lithography-etching-based fabrication is used to build electro-stimulation apparatus (10). Etching/lithography can be used to create micro needle electrodes (18) and arrays.

In another example, direct micromilling is used to fabricate the electro-stimulation apparatus (10). Micro-scale cutting tools (micro-endmills and micro-drills) as small as 10 μm diameter can be used to create features on high-precision CNC machines. Those machines are generally specifically designed for micromachining, including ultra-precision motion stages and ultra-high-speed (e.g., 80,000 to 160,000 rpm) spindles. This is another method to structure the electro-stimulation apparatus (10). One advantage of this method is that the base (13) can already be the desired thickness and may eliminate the need for attaching another conductive layer.

In another variation of the invention, horizontal arrays and assembly can be used. Similar to the bent-out needle approach, a horizontal (2D) array of micro needle electrode (18) can be created using micromilling, laser milling, or etching. Some of the challenges (e.g., tip sharpening) of this approach are thus similar to those for the bent-out needle approach. However, one advantage here is that no bending is required, eliminating fixtures for this purpose. Conversely, the fabricated 2D arrays will need to be assembled onto the base (13) with slits and conductive adhesives.

In another variation of the invention, a high-precision micromachining process that can be used for fabricating the electro-stimulation apparatus (10) is diamond or CBN microturning, or Swiss turning. In general, microturning uses diamond, carbide, or ceramic tools that can be sharpened down to 100 nm edge radius, since low cutting forces will be required to ensure the straightness of the micro needle electrodes (18).

In a nutshell, the present invention proposes an electro-stimulation apparatus (10) comprising a plurality of micro needle electrodes (18), said micro needle electrodes (18) being effective in enabling sending and receiving of electric signals to stimulate the supplementary motor area, cerebellum, premotor area and/or subthalamic nucleus of a human. These micro needle electrodes (18) are attached to intrinsic auricular muscles such as helicis major muscles, helicis minor muscles, tragicus muscles and anti-tragicus muscles. The signal for stimulating the supplementary motor area, cerebellum, premotor area and/or subthalamic nucleus is produced by a control unit and fed directly to the micro needle electrodes (18).

The adjustments to the stimulation signal can typically be carried out by changing the amplitude, frequency, pulse width, and pulse shape such as the harmonic content of the periodic pulses etc.

The electro-stimulation apparatus (10) typically comprises a communication unit 20 in signal communication with the control unit 19, enabling communication with other devices such as remote control units, computers, peripheral measurement/sensor units etc. The communication unit conventionally supports known communication protocols/standards (IR, USB, IEEE 802 family, Bluetooth, RF communication interface, RS-232, RS-422, RS-485, SPI (serial peripheral interface) i2c, as well as proprietary interfaces and/or protocols etc.).

In an example, an electro-stimulation apparatus (10) comprises at least one micro needle electrode (18) having stimulation end (11) and a base (13), said at least one micro needle electrode (18) being provided with a stimulation end (11) configured to stimulate intrinsic auricular muscles of a human and said stimulation end (11) of said micro needle electrode (18) is adapted to generate an electrical stimulation signal during a stimulating state

In a further example, said micro needle electrodes (18) are configured to be detachable from the base (13).

In a still further example, said micro needle electrodes (18) are fixedly attached to the base (13).

In a still further example, said base (13) enables any electrical leads attachment onto the back of the assembly through a conductive backing (14).

In a still further example, said base (13) and said conductive backing (14) comprise a magnetic element (12) between them.

In a still further example, said micro needle electrodes (18) form undercuts (15) to retain said stimulation ends (11) in place.

In a still further example, said undercut (15) has a dissolvable undercut element (17).

In a still further example, said dissolvable undercut element (17) comprises dissoluble materials.

In a still further example, said dissolvable undercut element (17) expands said undercut (15) dimensions.

In a still further example, a whole body of said undercut (15) comprises said dissolvable undercut element (17).

In a still further example, said dissolvable undercut element (17) comprises PLGA in which the dissolution time can be selected.

In a still further example, said dissolvable undercut element (17) comprises said anti-inflammatory or local anesthetics or painkiller substances.

In a still further example, said base (13) comprises an adhesive layer (16) extending on the surface of said base (13).

In a still further example, the signal produced by the control unit (19) has a voltage of 0V-15V and the frequency thereof is between 2 Hz-250 Hz.

In a still further example, said micro needle electrodes (18) having a height of 0.1-6 mm.

In a still further example, said micro needle electrodes (18) having a diameter of 50-750 μm.

In a still further example, said dissolvable undercut element (17) comprises dissolvable materials that dissolve with the exposure of electrical stimulation frequency.

In a still further examples, said dissolvable materials comprising hydrogen sulfide or levodopa.

The methods, devices, processing, circuitry, and logic described above may be implemented in many different ways and in many different combinations of hardware and software. For example, all or parts of the control unit (19) and/or communication unit (20) implementations may be circuitry that includes an instruction processor, such as a Central Processing Unit (CPU), microcontroller, or a microprocessor; or as an Application Specific Integrated Circuit (ASIC), Programmable Logic Device (PLD), or Field Programmable Gate Array (FPGA); or as circuitry that includes discrete logic or other circuit components, including analog circuit components, digital circuit components or both; or any combination thereof. The circuitry may include discrete interconnected hardware components or may be combined on a single integrated circuit die, distributed among multiple integrated circuit dies, or implemented in a Multiple Chip Module (MCM) of multiple integrated circuit dies in a common package, as examples.

Accordingly, the circuitry may store or access instructions for execution, or may implement its functionality in hardware alone. The instructions may be stored in a tangible storage medium that is other than a transitory signal, such as a flash memory, a Random Access Memory (RAM), a Read Only Memory (ROM), an Erasable Programmable Read Only Memory (EPROM); or on a magnetic or optical disc, such as a Compact Disc Read Only Memory (CDROM), Hard Disk Drive (HDD), or other magnetic or optical disk; or in or on another machine-readable medium. A product, such as a computer program product, may include a storage medium and instructions stored in or on the medium, and the instructions when executed by the circuitry in a device may cause the device to implement any of the processing described above or illustrated in the drawings.

The implementations may be distributed. For instance, the circuitry may include multiple distinct system components, such as multiple processors and memories, and may span multiple distributed processing systems. Parameters, databases, and other data structures may be separately stored and managed, may be incorporated into a single memory or database, may be logically and physically organized in many different ways, and may be implemented in many different ways. Example implementations include linked lists, program variables, hash tables, arrays, records (e.g., database records), objects, and implicit storage mechanisms. Instructions may form parts (e.g., subroutines or other code sections) of a single program, may form multiple separate programs, may be distributed across multiple memories and processors, and may be implemented in many different ways. Example implementations include stand-alone programs, and as part of a library, such as a shared library like a Dynamic Link Library (DLL). The library, for example, may contain shared data and one or more shared programs that include instructions that perform any of the processing described above or illustrated in the drawings, when executed by the circuitry.

In some examples, each unit, subunit, and/or module of the system may include a logical component. Each logical component may be hardware or a combination of hardware and software. For example, each logical component may include an application specific integrated circuit (ASIC), a Field Programmable Gate Array (FPGA), a digital logic circuit, an analog circuit, a combination of discrete circuits, gates, or any other type of hardware or combination thereof. Alternatively or in addition, each logical component may include memory hardware, such as a portion of the memory, for example, that comprises instructions executable with the processor or other processors to implement one or more of the features of the logical components. When any one of the logical components includes the portion of the memory that comprises instructions executable with the processor, the logical component may or may not include the processor. In some examples, each logical components may just be the portion of the memory or other physical memory that comprises instructions executable with the processor or other processor to implement the features of the corresponding logical component without the logical component including any other hardware. Because each logical component includes at least some hardware even when the included hardware comprises software, each logical component may be interchangeably referred to as a hardware logical component.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, ... <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

1. An electro-stimulation apparatus comprising:
at least one micro needle electrode having a stimulation end and a base,
said at least one micro needle electrode being provided with the stimulation end configured to stimulate intrinsic auricular muscles of a human and
said stimulation end of said micro needle electrode is adapted to generate an electrical stimulation signal during a stimulating state;
said micro needle electrode being detachably coupled with the base by a mechanical connection.

2. An electro-stimulation apparatus as in claim 1, wherein said micro needle electrode is fixedly attached to the base by the mechanical connection.

3. An electro-stimulation apparatus as in claim 1, wherein said base enables detachable coupling of electrical leads onto a back surface of the base through a conductive backing, said micro needle electrode extending away from a front surface of the base opposite the back surface of the base.

4. An electro-stimulation apparatus as in claim 3, further comprising a magnetic element disposed between said base and said conductive backing.

5. An electro-stimulation apparatus as in claim 1, wherein said micro needle electrodes include undercuts proximate said simulation ends to retain said stimulation ends in contact with said intrinsic auricular muscles of said human.

6. An electro-stimulation apparatus as in claim 5, wherein said undercut comprises a dissolvable undercut element.

7. An electro-stimulation apparatus as in claim 6, wherein said dissolvable undercut element comprises dissolvable materials.

8. An electro-stimulation apparatus as in claim 6, wherein said dissolvable undercut element is included in said undercut such that said undercut is expanded in at least one outer envelope dimension by said dissolvable undercut element.

9. An electro-stimulation apparatus as in claim 6, wherein a whole body of said undercut is formed with said dissolvable undercut element.

10. An electro-stimulation apparatus as in claim 6, wherein said dissolvable undercut element comprises polylactic-co-glycolic acid (PLGA) in which a predetermined dissolution time is selected.

11. An electro-stimulation apparatus as in claim 6, wherein said dissolvable undercut element encapsulates an anti-inflammatory, or local anesthetics, or painkiller substances or combinations thereof.

12. An electro-stimulation apparatus as in claim 1, wherein said base comprises an adhesive layer extending on a surface of said base.

13. An electro-stimulation apparatus as in claim 1, wherein the electrical stimulation signal is produced by a control unit circuitry.

14. An electro-stimulation apparatus as in claim 13, wherein the control unit circuitry is configured to produce the electrical stimulation signal with a voltage between 0V-15V and a frequency between 2 Hz-250 Hz.

15. An electro-stimulation apparatus as in claim 1, wherein said micro needle electrodes extend away from said base (13) to a distance of between 0.1-6 mm.

16. An electro-stimulation apparatus as in claim 1, wherein said micro needle electrodes have a diameter between 50-750 μm.

17. An electro-stimulation apparatus as in claim 8, wherein said dissolvable undercut element comprises dissolvable materials that dissolve in response to exposure to an electrical stimulation.

18. An electro-stimulation apparatus as in claim 8, wherein said dissolvable materials comprise hydrogen sulfide or levodopa.

19. An electro-stimulation apparatus comprising:
a base made of conductive material; and
a micro needle electrode mechanically assembled with the base to enable disassembly and replacement of the micro needle electrode;
the micro needle electrode comprising a stimulation end opposite the base, the stimulation end configured for insertion through auricular skin of a human to stimulate intrinsic auricular muscles of the human;
the stimulation end of said micro needle electrode configured to conduct an electrical stimulation signal into the intrinsic auricular muscles of the human during a stimulating state, wherein the base is configured to remain on top of the auricular skin and the electrical stimulation signal is conducted through the base to the micro needle electrode.

20. The electro-stimulation apparatus of claim 19 further comprising a dissolvable material coated or molded at the stimulation end of the micro needle electrode, the dissolvable material formed on the stimulation end of the micro needle electrode to hold the micro needle electrode in the auricular tissue of the human.

21. The electro-stimulation apparatus of claim 19, further comprising a conductive backing on a side of the base that is opposite a side of the base mechanically assembled with the micro needle electrode, the conductive backing being magnetic and enabling magnetic attachment of an electrical lead to the base to form an electrical connection therebetween and enable supply, by the electrical lead, of the electrical stimulation signal to the base.
